(19) United States
(12) Patent Application Publication
(10) Pub. No.: US 2026/0083675 A1
(43) Pub. Date: Mar. 26, 2026

(54) HIGH-BIOACTIVITY GOAT COLOSTRUM COMPOSITE FREEZE-DRIED POWDER AND PREPARATION METHOD THEREOF

(71) Applicant: Zishenghuo (Hunan) Food Research Institute Co., Ltd., Changsha (CN)

(72) Inventors: Boxing Chen, Changsha (CN); Lei Zhang, Changsha (CN)

(21) Appl. No.: 18/923,357

(22) Filed: Oct. 22, 2024

(30) Foreign Application Priority Data
Sep. 23, 2024 (CN) 2024113245782

(51) Int. Cl. A61K 9/127; A61K 9/19; A61K 35/20; A61K 35/745; A61K 35/747; A61K 47/26

(52) U.S. Cl. CPC A61K 9/1277; A61K 9/19; A61K 35/20; A61K 35/745; A61K 35/747; A61K 47/26

(57) ABSTRACT

A high-bioactivity goat colostrum composite freeze-dried powder relates to the technical field of food, comprising the following components in parts by weight: 60-70 parts of goat colostrum powder, 5-8 parts of composite probiotic liposome freeze-dried powder, and 6-9 parts of mannose oligosaccharides; the composite probiotic liposome freeze-dried powder is prepared from the following components: gallic acid, phytosterol, soybean phosphatidylcholine, modified chitosan, and probiotic powder; the probiotic powder is Bifidobacterium longum powder or Lactobacillus acidophilus powder. The invention proposes to prepare modified phytosterol by esterification reaction of gallic acid and phytosterol, and to prepare probiotic liposomes by encapsulating probiotics with the modified phytosterol and soybean phosphatidylcholine, and at the same time introduce carboxymethyl into chitosan to prepare modified chitosan, and to prepare composite probiotic liposome freeze-dried powder by encapsulating probiotic liposomes with the modified chitosan, thereby protecting probiotics, thereby reducing the inactivation of probiotics, and improving their survival rate.

### 1. TECHNICAL FIELD

The invention relates to the technical field of food, in particular to a high-bioactivity goat colostrum composite freeze-dried powder and a preparation method thereof.

### 2. BACKGROUND ART

Probiotics, as a group of active microorganisms in the human intestine that are beneficial to health, encompass a diverse range of types, including lactic acid bacteria such as bifidobacteria, lactobacilli, streptococci, and enterococci, as well as non-lactic acid bacteria such as bacilli, yeasts, and propionibacteria. These microorganisms play a crucial role in the human body. They not only facilitate the digestion and absorption of nutrients, but also significantly enhance immune function, helping to maintain the balanced state of intestinal flora. Furthermore, probiotics also possess important functions such as enhancing antioxidant capacity, inhibiting intestinal inflammation, lowering serum cholesterol levels, and protecting the intestinal mucosal barrier. However, currently, probiotic products available on the market can suffer from inactivation of probiotics due to environmental changes during processing, transportation, storage, as well as digestion and absorption. The number of probiotics that can actually colonize the intestinal tract in a live state is insufficient to exert their probiotic effects. Therefore, ensuring that probiotics colonize the human intestinal tract with high activity and sufficient quantity is a problem that needs to be addressed currently.

The existing technology has the following main problems: it is difficult for probiotics to maintain high activity and sufficient quantity to enter the human intestine.

### 3. SUMMARY OF THE INVENTION

In view of the above situation, in order to overcome the defects of the prior art, the invention provides a high-bioactivity goat colostrum composite freeze-dried powder and a preparation method thereof. In order to solve the problem that it is difficult for probiotics to maintain high activity and sufficient quantity to enter the human intestine, the invention proposes to prepare modified phytosterol by esterification reaction of gallic acid and phytosterol, and to prepare probiotic liposomes by encapsulating probiotics with the modified phytosterol and soybean phosphatidylcholine, and at the same time introduce carboxymethyl into chitosan to prepare modified chitosan, and to prepare composite probiotic liposome freeze-dried powder by encapsulating probiotic liposomes with the modified chitosan, thereby protecting probiotics, thereby reducing the inactivation of probiotics, and improving their survival rate and colonization number in the intestine.

In order to realize the above objects, the invention adopts the following technical scheme: a high-bioactivity goat colostrum composite freeze-dried powder comprises the following components in parts by weight: 60-70 parts of goat colostrum powder, 5-8 parts of composite probiotic liposome freeze-dried powder, and 6-9 parts of mannose oligosaccharides; the composite probiotic liposome freeze-dried powder is prepared from the following components: gallic acid, phytosterol, soybean phosphatidylcholine, modified chitosan, and probiotic powder; the probiotic powder is Bifidobacterium longum powder or Lactobacillus acidophilus powder.

Further, the preparation method of the modified chitosan comprises the following steps:
S1, weighing chloroacetic acid and adding it to isopropanol, the amount of the chloroacetic acid added to the isopropanol is 100 mg/mL, stirring until completely dissolved to obtain a chloroacetic acid-isopropanol solution;
S2, weighing chitosan and adding it to isopropanol, stirring for 1-2 hours to obtain a mixed solution, then adding sodium hydroxide solution, stirring in a constant temperature water bath, slowly adding the chloroacetic acid-isopropanol solution prepared in step S1, reacting for 2-3 hours, adjusting the pH value to 7, washing, and drying to obtain the modified chitosan.

Preferably, in step S2, the amount of the chitosan added to the isopropanol is 100-150 mg/mL, the volume ratio of the mixed solution to the sodium hydroxide solution is 2:3, and the volume ratio of the mixed solution to the chloroacetic acid-isopropanol solution is 1:1-1.5.

Preferably, in step S2, the sodium hydroxide solution is a solution of sodium hydroxide in water, the mass fraction of the sodium hydroxide solution is 30%, the stirring temperature of the constant temperature water bath is 50-60° C., and the stirring speed is 100-200 rpm.

Further, the preparation method of the composite probiotic liposome freeze-dried powder comprises the following steps:
(1) weighing a certain amount of gallic acid and phytosterol and dissolving them in a reaction solvent, adding a catalyst, placing the mixture in a thermostat at 50° C. for 10-20 hours, separating and purifying the obtained product to obtain modified phytosterol;
(2) weighing a certain amount of soybean phosphatidylcholine, and dissolving it and the modified phytosterol prepared in step (1) in a chloroform-methanol mixture with a volume ratio of 1:1, placing them in the dark at 25° C. for 15-30 minutes, removing the chloroform-methanol mixture by rotary evaporation, adding a certain amount of phosphate buffered saline solution and probiotic powder, and performing ultrasonic treatment in a constant temperature water bath to obtain probiotic liposomes;
(3) weighing a certain amount of modified chitosan and dissolving it in water, adding a certain amount of the probiotic liposomes prepared in step (2), stirring in a constant temperature water bath, performing ultrasonic treatment, and freeze-drying to obtain the composite probiotic liposome freeze-dried powder.

Preferably, in step (1), the molar ratio of the gallic acid to the phytosterol is 1:1.

Preferably, in step (1), the catalyst is p-toluenesulfonic acid, the molar ratio of the catalyst to the phytosterol is 1:0.1, and the reaction solvent is toluene or tetrahydrofuran.

Preferably, in step (2), the weight ratio of the probiotic powder, the soybean phosphatidylcholine and the modified phytosterol is 1:8-10:2-4, the temperature of the constant temperature water bath is 40-50° C., the time of the ultrasonic treatment is 30-40 min, the power is 200 W, the volume ratio of the phosphate buffered saline solution to the organic solvent is 5-6:3, and the pH value of the phosphate buffered saline solution is 7.

Preferably, in step (3), the amount of the modified chitosan added to the water is 100-150 mg/mL, the mass ratio of the modified chitosan to the probiotic liposomes is 10:1, the stirring temperature of the constant temperature water bath is 40-50° C., the stirring speed is 100-200 rpm, the ultrasonic treatment time is 10-20 min, and the power is 200 W.

The invention provides a preparation method of the high-bioactivity goat colostrum composite freeze-dried powder, comprising the following steps: adding the composite probiotic liposome freeze-dried powder and the mannose oligosaccharides to the goat colostrum powder, stirring evenly to obtain the high-bioactivity goat colostrum composite freeze-dried powder.

The invention has the following advantageous effects:
the invention proposes to prepare modified phytosterol by esterification reaction of gallic acid and phytosterol, and to prepare probiotic liposomes by encapsulating probiotics with the modified phytosterol and soybean phosphatidylcholine, and at the same time introduce carboxymethyl into chitosan to prepare modified chitosan, and to prepare composite probiotic liposome freeze-dried powder by encapsulating probiotic liposomes with the modified chitosan, thereby protecting probiotics, thereby reducing the inactivation of probiotics, and improving their survival rate and colonization number in the intestine, so as to truly exert its probiotic effect. The wall materials of conventional liposomes are primarily composed of phospholipids and cholesterol; however, cholesterol is not suitable for individuals with cardiovascular diseases. Phytosterol, which possesses a chemical structure similar to cholesterol, can be used as a substitute to enhance the stability and encapsulation efficiency of liposomes; modifying phytosterol with gallic acid can further boost its antioxidant effect, preventing the oxidation and deterioration of the composite probiotic liposome freeze-dried powder during processing, storage, and transportation, thereby improving the storage stability of the composite probiotic liposome freeze-dried powder. Introducing carboxymethyl into the molecular structure of chitosan to prepare modified chitosan can enhance the water solubility of chitosan, thereby facilitating the subsequent preparation of the composite probiotic liposome freeze-dried powder. Since liposomes have potential risks of damage during the freeze-drying process, encapsulating probiotic liposomes with the modified chitosan to prepare composite probiotic liposome freeze-dried powder can protect the probiotic liposomes during freeze-drying, thereby reducing the impact of freeze-drying on the probiotic liposomes. Mannose oligosaccharides can promote the proliferation of intestinal probiotics in the body and improve human intestinal function.

### 4. BRIEF DESCRIPTION OF ACCOMPANY DRAWINGS

FIG. 1 is a graph showing the encapsulation efficiency test results of the composite probiotic liposome freeze-dried powder prepared in Embodiments 1-3 and Comparative Examples 1 and 2 of the invention.

FIG. 2 is a graph showing the storage stability test results of the composite probiotic liposome freeze-dried powder prepared by Embodiments 1-3 and Comparative Examples 1 and 2 of the invention.

FIG. 3 is a graph showing the simulated gastric fluid test results of the composite probiotic liposome freeze-dried powder prepared in Embodiments 1-3 and Comparative Examples 1 and 2 of the invention.

FIG. 4 is a graph showing the simulated digestion test results of the composite probiotic liposome freeze-dried powder prepared in Embodiments 1-3 of the invention.

FIG. 5 is a graph showing the redispersibility test results of the composite probiotic liposome freeze-dried powder prepared in Embodiments 1-3 of the invention.

FIG. 6 shows a TEM image of the composite probiotic liposome freeze-dried powder prepared in Embodiment 1.

The accompanying drawings are used to provide a further understanding of the invention and constitute a part of the specification; together with the embodiments of the invention, they are used to explain the invention and do not constitute a limitation of the invention.

### 5. Specific Embodiment of the Invention

The technical scheme in the embodiments of the invention will be described clearly and completely below in combination with the accompanying drawings in the embodiments of the invention. Obviously, the embodiments are only some of the embodiments of the invention, rather than all the embodiments. Based on the embodiments of the invention, all other embodiments obtained by ordinary technicians in this field without making creative work are within the protection scope of the invention.

Unless otherwise defined, all professional and scientific terms used herein have the same meanings as those familiar to those skilled in the art. In addition, any methods and materials similar or equivalent to those described herein can be applied to the invention. The preferred embodiments and materials described herein are for demonstration purposes only and do not limit the content of the invention.

The experimental methods in the following embodiments, unless otherwise specified, are all conventional methods; the experimental materials and test strains used in the following embodiments, unless otherwise specified, are all purchased from commercial channels.

### Embodiment 1

A high-bioactivity goat colostrum composite freeze-dried powder comprises the following components in parts by weight: 60 parts of goat colostrum powder, 5 parts of composite probiotic liposome freeze-dried powder, and 6 parts of mannose oligosaccharides. The preparation method of the modified chitosan comprises the following steps:
S1, weighing 3 g of chloroacetic acid and adding it to 30 mL of isopropanol, stirring until completely dissolved to obtain a chloroacetic acid-isopropanol solution;
S2, weighing 2 g of chitosan and adding it to 20 mL of isopropanol, stirring for 1 hour to obtain a mixed solution, then adding 30 mL of 30% sodium hydroxide solution, stirring in a 50° C. constant temperature water bath at a speed of 100 rpm, slowly adding 20 mL of the chloroacetic acid-isopropanol solution prepared in step S1, reacting for 2 hours, adjusting the pH value to 7, washing, and drying to obtain the modified chitosan.

The preparation method of the composite probiotic liposome freeze-dried powder comprises the following steps:
(1) weighing 10 mmol of gallic acid and 10 mmol of phytosterol and dissolving them in 50 mL of toluene, adding 1 mmol of p-toluenesulfonic acid, placing the mixture in a thermostat at 50° C. for 10 hours, separating and purifying the obtained product to obtain modified phytosterol;
(2) weighing 0.8 g of soybean phosphatidylcholine, and dissolving it and 0.2 g of the modified phytosterol prepared in step (1) in 30 mL of a chloroform-methanol mixture with a volume ratio of 1:1, placing them in the dark at 25° C. for 15 minutes, removing the chloroform-methanol mixture by rotary evaporation, adding 50 mL of phosphate buffered saline solution (pH 7) and 0.1 g of Bifidobacterium longum powder, and performing ultrasonic treatment for 30 min with an ultrasonic power of 200 W in a 40° C. constant temperature water bath to obtain probiotic liposomes;
(3) weighing 5 g of modified chitosan and dissolving it in 50 mL of water, adding 0.5 g of the probiotic liposomes prepared in step (2), stirring in a 40° C. constant temperature water bath at a speed of 100 rpm, performing ultrasonic treatment for 10 min at a power of 200 W, and freeze-drying to obtain the composite probiotic liposome freeze-dried powder.

The embodiment provides a preparation method of the high-bioactivity goat colostrum composite freeze-dried powder, comprising the following steps: adding the composite probiotic liposome freeze-dried powder and the mannose oligosaccharides to the goat colostrum powder in parts by weight, stirring evenly to obtain a high-bioactivity goat colostrum composite freeze-dried powder.

### Embodiment 2

A high-bioactivity goat colostrum composite freeze-dried powder comprises the following components in parts by weight: 70 parts of goat colostrum powder, 8 parts of composite probiotic liposome freeze-dried powder, and 9 parts of mannose oligosaccharides.

The preparation method of the modified chitosan comprises the following steps:

S1, weighing 3 g of chloroacetic acid and adding it to 30 mL of isopropanol, stirring until completely dissolved to obtain a chloroacetic acid-isopropanol solution;

S2, weighing 3 g of chitosan and adding it to 20 mL of isopropanol, stirring for 2 hours to obtain a mixed solution, then adding 30 mL of 30% sodium hydroxide solution, stirring in a 60° C. constant temperature water bath at a speed of 200 rpm, slowly adding 30 mL of the chloroacetic acid-isopropanol solution prepared in step S1, reacting for 3 hours, adjusting the pH value to 7, washing, and drying to obtain the modified chitosan.

The preparation method of the composite probiotic liposome freeze-dried powder comprises the following steps:
(1) weighing 10 mmol of gallic acid and 10 mmol of phytosterol and dissolving them in 50 mL of tetrahydrofuran, adding 1 mmol of p-toluenesulfonic acid, placing the mixture in a thermostat at 50° C. for 20 hours, separating and purifying the obtained product to obtain modified phytosterol;
(2) weighing 1 g of soybean phosphatidylcholine, and dissolving it and 0.4 g of the modified phytosterol prepared in step (1) in 30 mL of a chloroform-methanol mixture with a volume ratio of 1:1, placing them in the dark at 25° C. for 30 minutes, removing the chloroform-methanol mixture by rotary evaporation, adding 60 mL of phosphate buffered saline solution (pH 7) and 0.1 g of Lactobacillus acidophilus powder, and performing ultrasonic treatment for 40 min with an ultrasonic power of 200 W in a 50° C. constant temperature water bath to obtain probiotic liposomes;
(3) weighing 7.5 g of modified chitosan and dissolving it in 50mL of water, adding 0.75 g of the probiotic liposomes prepared in step (2), stirring in a 50° C. constant temperature water bath a speed of 200 rpm, performing ultrasonic treatment for 20 min at a power of 200 W, and freeze-drying to obtain the composite probiotic liposome freeze-dried powder.

The embodiment provides a preparation method of the high-bioactivity goat colostrum composite freeze-dried powder, comprising the following steps: adding the composite probiotic liposome freeze-dried powder and the mannose oligosaccharides to the goat colostrum powder in parts by weight, stirring evenly to obtain a high-bioactivity goat colostrum composite freeze-dried powder.

### Embodiment 3

A high-bioactivity goat colostrum composite freeze-dried powder comprises the following components in parts by weight: 65 parts of goat colostrum powder, 7 parts of composite probiotic liposome freeze-dried powder, and 8 parts of mannose oligosaccharides.

The preparation method of the modified chitosan comprises the following steps:

S1, weighing 3 g of chloroacetic acid and adding it to 30 mL of isopropanol, stirring until completely dissolved to obtain a chloroacetic acid-isopropanol solution;

S2, weighing 2.5 g of chitosan and adding it to 20 mL of isopropanol, stirring for 1.5 hours to obtain a mixed solution, then adding 30 mL of 30% sodium hydroxide solution, stirring in a 55° C. constant temperature water bath at a speed of 150 rpm, slowly adding 25 mL of the chloroacetic acid-isopropanol solution prepared in step S1, reacting for 2.5 hours, adjusting the pH value to 7, washing, and drying to obtain the modified chitosan.

The preparation method of the composite probiotic liposome freeze-dried powder comprises the following steps:
(1) weighing 10 mmol of gallic acid and 10 mmol of phytosterol and dissolving them in 50 mL of toluene, adding 1 mmol of p-toluenesulfonic acid, placing the mixture in a thermostat at 50° C. for 15 hours, separating and purifying the obtained product to obtain modified phytosterol;
(2) weighing 0.9 g of soybean phosphatidylcholine, and dissolving it and 0.3 g of the modified phytosterol prepared in step (1) in 30 mL of a chloroform-methanol mixture with a volume ratio of 1:1, placing them in the dark at 25° C. for 20 minutes, removing the chloroform-methanol mixture by rotary evaporation, adding 55 mL of phosphate buffered saline solution (pH 7) and 0.1 g of Lactobacillus acidophilus powder, and performing ultrasonic treatment for 35 min with an ultrasonic power of 200 W in a 45° C. constant temperature water bath to obtain probiotic liposomes;
(3) weighing 7 g of modified chitosan and dissolving it in 50 mL of water, adding 0.7 g of the probiotic liposomes prepared in step (2), stirring in a 45° C. constant temperature water bath a speed of 150 rpm, performing ultrasonic treatment for 15 min at a power of 200 W, and freeze-drying to obtain the composite probiotic liposome freeze-dried powder.

The embodiment provides a preparation method of the high-bioactivity goat colostrum composite freeze-dried powder, comprising the following steps: adding the composite probiotic liposome freeze-dried powder and the mannose oligosaccharides to the goat colostrum powder in parts by weight, stirring evenly to obtain a high-bioactivity goat colostrum composite freeze-dried powder.

### Comparative Example 1

This comparative example provides a composite probiotic liposome freeze-dried powder, which differs from Embodiment 1 in that modified phytosterol is not prepared, and phytosterol is used instead of modified phytosterol, and the remaining components and component contents are the same as those in Embodiment 1.

### Comparative Example 2

This comparative example provides a composite probiotic liposome freeze-dried powder, which differs from Embodiment 1 in that the composite probiotic liposome freeze-dried powder does not contain modified chitosan, and the remaining components and component contents are the same as those in Embodiment 1.

### Experimental Example 1

The encapsulation efficiency of the composite probiotic liposome freeze-dried powder prepared in Embodiments 1-3 and Comparative Examples 1 and 2 of the invention was tested, with the specific method as follows: weighing the composite probiotic liposome freeze-dried powder and adding it to a mixture of methanol and distilled water, ultrasonicating at 400 W for 10-20 minutes, then centrifuging at 8000 r/min for 20 minutes to obtain microbial sludge, adding phosphate buffer solution to the bacterial sludge, adding lysozyme, and subjecting it to enzymatic hydrolysis at 40° C. for 60 minutes, performing ultrasonic treatment at 500 W for 30 minutes to obtain enzymatic hydrolysate, taking 1 mL of the enzymatic hydrolysate, adding 5 mL of Coomassie Brilliant Blue G-250 reagent, mixing well and letting it stand for 2 minutes; measuring the absorbance at 595 nm by using an ultraviolet-visible spectrophotometer to calculate the probiotic protein content A₁ encapsulated in liposomes, using the same method to determine the total probiotic protein content A₂, encapsulation efficiency=A₁/A₂×100%.

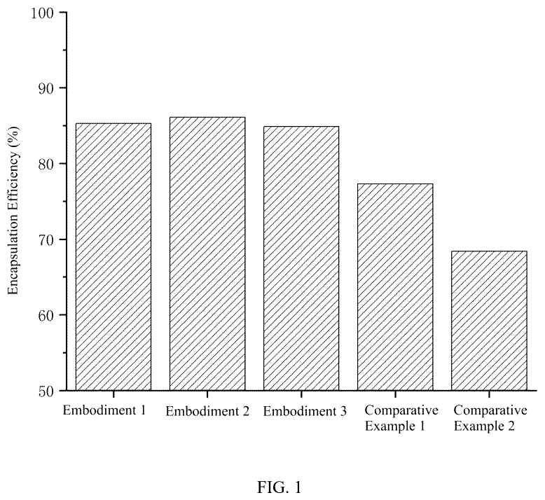

FIG. 1 is a graph showing the encapsulation efficiency test results of the composite probiotic liposome freeze-dried powder prepared in Embodiments 1-3 and Comparative Examples 1 and 2 of the invention. As shown in the figure, the encapsulation efficiency of Embodiments 1-3 is 85.3%, 86.1%, and 84.9%, respectively, and the encapsulation efficiency of Comparative Examples 1 and 2 is 77.3% and 68.4%, respectively. The encapsulation efficiency of Embodiments 1-3 is higher than that of Comparative Example 1, indicating that the modified phytosterol with the introduction of gallic acid can increase the encapsulation efficiency. The encapsulation efficiencies of Embodiments 1-3 are all higher than those of Comparative Example 2, indicating that the addition of modified chitosan increases the encapsulation efficiency and improves the protective effect of the composite probiotic liposome freeze-dried powder on the probiotics.

### Experimental Example 2

A storage stability test of the composite probiotic liposome freeze-dried powder prepared in Embodiments 1-3 and Comparative Examples 1 and 2 of the invention was performed as follows: the composite probiotic liposome freeze-dried powder was weighed and placed in a constant temperature and humidity chamber at a temperature of 30° C. and a humidity of 60%. After storage for 60 days, the encapsulation efficiency test was performed according to Experimental Example 1.

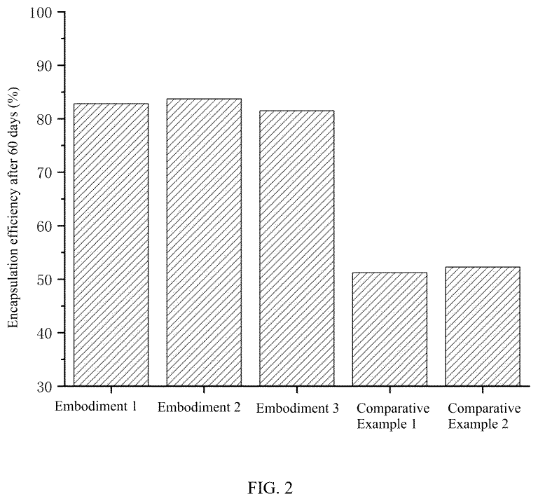

FIG. 2 is a graph showing the storage stability test results of the composite probiotic liposome freeze-dried powder prepared by Embodiments 1-3 and Comparative Examples 1 and 2 of the invention. As shown in the figure, the test results of Embodiments 1-3 are 82.8%, 83.7%, and 81.5%, respectively, and the test results of Comparative Examples 1 and 2 are 51.2% and 52.3%, respectively. Combining Experimental Example 1 and Experimental Example 2, the encapsulation efficiencies of Embodiments 1-3 change very little after storage for 60 days, but there is a significant decrease in Comparative Example 1, indicating that the storage stability of the composite probiotic liposome freeze-dried powder can be effectively improved after the modified plant sterol introduces gallic acid. The decrease in Comparative Example 2 is larger than that of Embodiments 1-3, indicating that the coating with modified chitosan can improve the storage stability of the composite probiotic liposome freeze-dried powder.

### Experimental Example 3

A simulated gastric fluid test was conducted on the composite probiotic liposome freeze-dried powder prepared in Embodiments 1-3 and Comparative Examples 1 and 2 of the invention. The specific method is as follows: 3 g of pepsin was dissolved in 1 L of a 0.5% NaCl solution, and the pH was adjusted to 2 to prepare artificial gastric fluid; the composite probiotic liposome freeze-dried powder prepared from Embodiments 1-3 and Comparative Examples 1-2 was added to the artificial gastric fluid; according to the gastric emptying time of 4 h, the mixture was shaken at 100 rpm under a 37° C. water bath condition; the probiotic content was measured at 0 h and 4 h, respectively; the content at 0 h was taken as the baseline value, and 4 h was taken as the final value; the preservation rate was calculated by the following formula: preservation rate=final value/baseline value×100%.

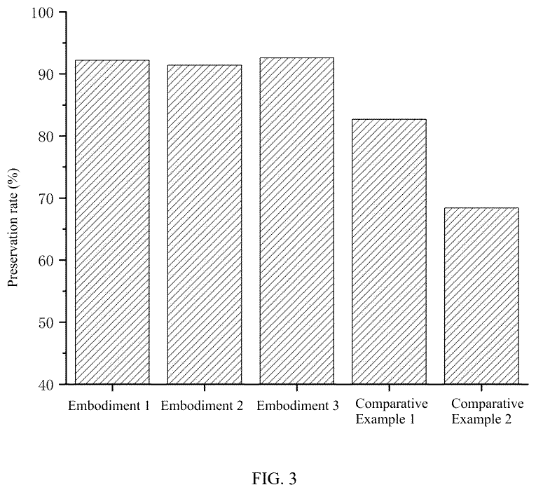

FIG. 3 is a graph showing the simulated gastric fluid test results of the composite probiotic liposome freeze-dried powder prepared in Embodiments 1-3 and Comparative Examples 1 and 2 of the invention. As shown in the figure, the preservation rates of probiotics in Embodiments 1-3 and Comparative Examples 1 and 2 are 92.2%, 91.4%, 92.6%, 82.7% and 68.4%, respectively; the preservation rate of probiotics in Embodiments 1-3 is significantly higher than that in Comparative Example 2, indicating that the coating with modified chitosan reduces the damage of gastric fluid to probiotics and plays a protective role.

### Experimental Example 4

A simulated digestion test was conducted on the composite probiotic liposome freeze-dried powders prepared in Embodiments 1-3. The specific method is as follows: 10 g of trypsin and 3 g of ox bile salt were dissolved in 1 L of a 0.5% NaCl solution, and the pH was adjusted to 7.5 to prepare artificial intestinal fluid; the composite probiotic liposome freeze-dried powder from Embodiments 1-3 was first subjected to a simulated gastric fluid test, and then added to the artificial intestinal fluid; the mixture was shaken at 37° C. and 100 rpm for 6 hours to simulate the intestinal digestion process; samples were taken at 0 hours and 6 hours to measure the probiotic content; the content at 0 h was taken as the baseline value, and the content at 6 h was taken as the final value; the release rate of probiotics was calculated by using the following formula: release rate=(baseline value-final value)/baseline value×100%.

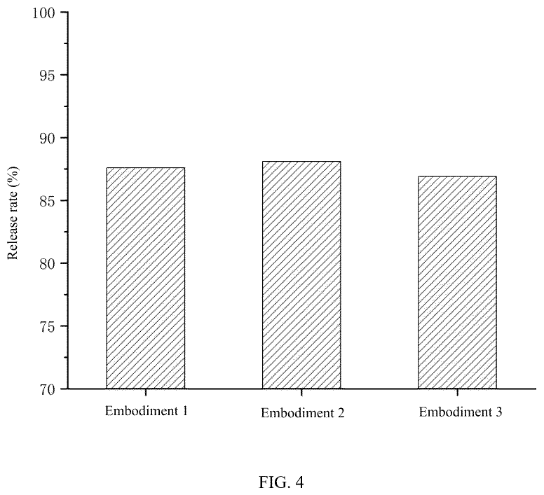

FIG. 4 is a graph showing the simulated digestion test results of the composite probiotic liposome freeze-dried powder prepared in Embodiments 1-3 of the invention. As shown in the figure, the release rates of Embodiments 1-3 are 87.6%, 88.1%, and 86.9%, respectively; the release rates of the probiotics in Embodiments 1-3 are all greater than 85%, indicating that the probiotics have a good release effect in the intestine.

### Experimental Example 5

A redispersibility test of the composite probiotic liposome freeze-dried powder prepared in Embodiments 1-3 was conducted. The specific method is as follows: the composite probiotic liposome freeze-dried powder prepared from Embodiments 1-3 was added to deionized water at 40° C. in an amount of 0.2 g/ml, and was stirred in one direction at 10 rpm for dissolution; the mixture was then filtered through a 60-mesh sieve; the dry weight of the filtered substance was taken as the agglomerated mass, the mass of the composite probiotic liposome freeze-dried powder added to the solution was taken as the total mass; the agglomeration rate was calculated by using the following formula: agglomeration rate=agglomerated mass/total mass×100%.

FIG. 5 is a graph showing the redispersibility test results of the composite probiotic liposome freeze-dried powder prepared in Embodiments 1-3 of the invention. As shown in the figure, the agglomeration rates of Embodiments 1-3 are 17.2%, 16.9%, and 16.3%, respectively, indicating that the composite probiotic liposome freeze-dried powder has good redispersibility.

### Experimental Example 6

The composite probiotic liposome freeze-dried powder prepared in Embodiment 1 was re-dissolved and observed under a transmission electron microscope (TEM) to examine its microstructure.

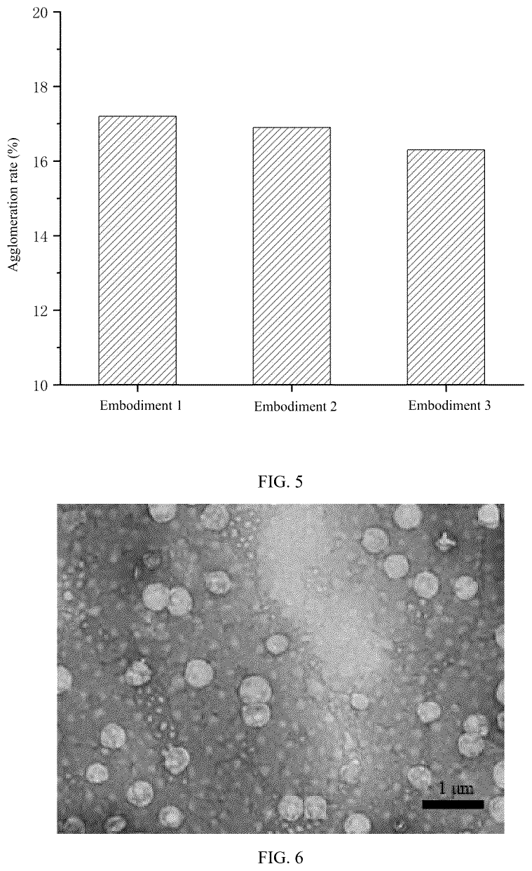

FIG. 6 shows a TEM image of the composite probiotic liposome freeze-dried powder prepared in Embodiment 1. From the figure, it can be seen that after re-dissolution, the freeze-dried powder appears rounded, with a smooth surface and uniform size.

Although the embodiments of the invention have been shown and described, it should be understood that those skilled in the art can make many changes, modifications, substitutions and alterations to the embodiments without departing from the principles and spirit of the invention.

The invention and its embodiments are described above, but this description is not restrictive. The embodiments shown in the accompanying drawings are only some of the embodiments of the invention, and the actual application is not limited thereto. In short, if ordinary technicians in this field are inspired by it and design methods and embodiments similar to the technical scheme without creativity without departing from the purpose of the invention, they should all fall within the protection scope of the invention.

1. A high-bioactivity goat colostrum composite freeze-dried powder, comprising the following components in parts by weight: 60-70 parts of goat colostrum powder, 5-8 parts of composite probiotic liposome freeze-dried powder, and 6-9 parts of mannose oligosaccharides; the composite probiotic liposome freeze-dried powder is prepared from the following components: gallic acid, phytosterol, soybean phosphatidylcholine, modified chitosan, and probiotic powder; the probiotic powder is Bifidobacterium longum powder or Lactobacillus acidophilus powder.

2. A preparation method of the high-bioactivity goat colostrum composite freeze-dried powder of claim 1, comprising the following steps: adding the composite probiotic liposome freeze-dried powder and the mannose oligosaccharides to the goat colostrum powder, stirring evenly to obtain the high-bioactivity goat colostrum composite freeze-dried powder.

3. The preparation method of the high-bioactivity goat colostrum composite freeze-dried powder of claim 2, wherein the preparation method of the modified chitosan comprises the following steps:
S1, weighing chloroacetic acid and adding it to isopropanol, wherein the amount of the chloroacetic acid added to the isopropanol is 100 mg/mL, stirring until completely dissolved to obtain a chloroacetic acid-isopropanol solution;
S2, weighing chitosan and adding it to isopropanol, stirring for 1-2 hours to obtain a mixed solution, then adding sodium hydroxide solution, stirring in a constant temperature water bath, slowly adding the chloroacetic acid-isopropanol solution prepared in step S1, reacting for 2-3 hours, adjusting the pH value to 7, washing, and drying to obtain the modified chitosan.

4. The preparation method of the high-bioactivity goat colostrum composite freeze-dried powder of claim 3, wherein in step S2, the amount of the chitosan added to the isopropanol is 100-150 mg/mL, the volume ratio of the mixed solution to the sodium hydroxide solution is 2:3, and the volume ratio of the mixed solution to the chloroacetic acid-isopropanol solution is 1:1-1.5.

5. The preparation method of the high-bioactivity goat colostrum composite freeze-dried powder of claim 3, wherein in step S2, the sodium hydroxide solution is a solution of sodium hydroxide in water, the mass fraction of the sodium hydroxide solution is 30%, the stirring temperature of the constant temperature water bath is 50-60° C., and the stirring speed is 100-200 rpm.

6. The preparation method of the high-bioactivity goat colostrum composite freeze-dried powder of claim 2, wherein the preparation method of the composite probiotic liposome freeze-dried powder comprises the following steps:
(1) weighing a certain amount of gallic acid and phytosterol and dissolving them in a reaction solvent, adding a catalyst, placing the mixture in a thermostat at 50° C. for 10-20 hours, separating and purifying the obtained product to obtain modified phytosterol;
(2) weighing a certain amount of soybean phosphatidylcholine, and dissolving it and the modified phytosterol prepared in step (1) in an organic solvent, hydrating them in the dark at 25° C. for 15-30 minutes, removing organic solvent by rotary evaporation, adding a certain amount of phosphate buffered saline solution and probiotic powder, and performing ultrasonic treatment in a constant temperature water bath to obtain probiotic liposomes;
(3) weighing a certain amount of modified chitosan and dissolving it in water, adding a certain amount of the probiotic liposomes prepared in step (2), stirring in a constant temperature water bath, performing ultrasonic treatment, and freeze-drying to obtain the composite probiotic liposome freeze-dried powder.

7. The preparation method of the high-bioactivity goat colostrum composite freeze-dried powder of claim 6, wherein in step (1), the molar ratio of the gallic acid to the phytosterol is 1:1.

8. The preparation method of the high-bioactivity goat colostrum composite freeze-dried powder of claim 6, wherein in step (1), the catalyst is p-toluenesulfonic acid, the molar ratio of the catalyst to the phytosterol is 1:0.1, and the reaction solvent is toluene or tetrahydrofuran.

9. The preparation method of the high-bioactivity goat colostrum composite freeze-dried powder of claim 6, wherein in step (2), the weight ratio of the probiotic powder, the soybean phosphatidylcholine and the modified phytosterol is 1:8-10:2-4, the temperature of the constant temperature water bath is 40-50° C., the time of the ultrasonic treatment is 30-40 min, the power is 200 W, the volume ratio of the phosphate buffered saline solution to the organic solvent is 5-6:3, and the pH value of the phosphate buffered saline solution is 7.

10. The preparation method of the high-bioactivity goat colostrum composite freeze-dried powder of claim 6, wherein in step (3), the amount of the modified chitosan added to the water is 100-150 mg/mL, the mass ratio of the modified chitosan to the probiotic liposomes is 10:1, the stirring temperature of the constant temperature water bath is 40-50° C., the stirring speed is 100-200 rpm, the ultrasonic treatment time is 10-20 min, and the power is 200 W.
